(19) United States
(12) Patent Application Publication
(10) Pub. No.: US 2022/0135933 A1
(43) Pub. Date: May 5, 2022

(54) PARALLEL AND INTERCONNECTED CELL CULTURE VESSEL SYSTEM

(71) Applicant: Scientific Industries, Inc., Bohemia, NY (US)

(72) Inventors: Joseph G. Cremonese, Greensburg, PA (US); Scott Anderson, Pittsburgh, PA (US); Kiersten Bradnam, Pittsburgh, PA (US); Zachary Guy Marino, Pittsburgh, PA (US); Abbie Underhill, Pittsburgh, PA (US)

(21) Appl. No.: 17/499,036

(22) Filed: Oct. 12, 2021

Related U.S. Application Data
(60) Provisional application No. 63/107,187, filed on Oct. 29, 2020.

(51) Int. Cl. C12N 1/04; C12M 1/36; C12M 1/34; C12M 1/00; C12M 1/12; C12M 3/00

(52) U.S. Cl. CPC C12N 1/04; C12M 41/48; C12M 41/26; C12M 23/48; C12M 29/10; C12M 25/14; C12M 41/34

(57) ABSTRACT

Embodiments relate to a unique design of a cell growth vessel stack system that provides for inter-communication of cell growth levels arranged in a parallel configuration so to facilitate environmental uniformity of the inter-connected growth surface tiers. The unique design also facilitates a fine-tuning of optimized gas and media flow to each cell growth level within the vessel stack. The unique architecture facilitates media refreshment at conveniently scheduled intervals and/or allows constant perfusion from a media reservoir that can be replenished without interrupting the cell proliferation rate. The perfusion rate, nutrient medium condition, CO2 content and osmolality can be controlled to optimize the desired cell proliferation rate, thereby improving cell quantity, quality, and viability.

### CROSS REFERENCE TO RELATED APPLICATIONS

This application claims the benefit of U.S. Prov. Appl. No. 63/107,187, filed Oct. 29, 2020 which is incorporated herein by reference in its entirety.

### FIELD OF THE INVENTION

The embodiments disclosed herein relate to a system of parallel and interconnected cell culture vessels which provides uniformity of growth environment while facilitating individual control of each vessel.

### BACKGROUND OF THE INVENTION

In the last few years, the Cell Therapy Industry (CTI) has emerged with cell production process requirements that have outpaced the upstream capabilities for developing the robust cell populations that will be required for scale-up seeding of production-volume reactors. The upstream limitations have, for the most part, been due to the limitations of the existing cell production and differentiating device designs that have not kept pace with the advancements in the technology of cell line development and in the growth of robust cell populations.

The CTI demands for treating millions of patients with therapeutic cells, over the next decade, will require a constant supply of cells increasing in need to many trillions of cells annually. Coupled with that, there will be a demand to expand cell production to include a variety of different cells and proteins for individualized treatment of specifically identified abnormal and perhaps life-threatening conditions. To satisfy such required demands, upstream cell culturing must be able to produce many different cell types in a reproducibly consistent process. To achieve that end, the upstream vessels must be designed to include sensors to enable real-time in-process monitoring and control of the cellular metabolic and physiological processes.

The original Nunc Cell Factory™ was designed about forty years ago and it has been improved and expanded in capacity several times since then. The Corning Cell Stack® is similar to the original Cell Factory and it came along after the original Cell Factory patents expired. Both the Cell Factory and Cell Stack are designed, architecturally, similar to a multi-tier auto parking garage with each level communicating serially and sequentially. Both Cell Factory and Cell Stack were designed to increase the total growth area for adherent cells by increasing the number of cell growth levels and, at the same time, for providing a culture environment that is simultaneously shared by the multi-tiered stack of cell growth surfaces. The novel idea being that a multilevel design, with each level serially connected, would provide inter-level conditions that were very similar on each level. The inter-level similarity of the cell growth environment was, to a great extent, effective for growing cells under similar and perhaps reproducible conditions.

The Cell Factory and Cell Stack design, effective in some respects, was deficient in other respects. The arrangement of stacked and interconnecting growth levels does not lend itself to the placement of optical sensors that could be installed in a location such that the sensors could receive an excitation signal from an external source and, in return, emit a signal that could be transmitted to an external receiver and interpreted. Such an arrangement would enable signal monitoring and use of the information garnered to control gas and nutrient media flow to the cell growth surfaces in response to the metabolic and physiological requirements for robust cell growth. The inventive design herein described is for a cell culture vessel stack that would satisfy the monitoring and control elements and, at the same time, rectify the absence of the design elements necessary to permit the utility of monitoring and control of the cell growth process.

### SUMMARY OF THE INVENTION

The application herein described discloses the unique design of a cell growth vessel stack system that provides for inter-communication of cell growth levels arranged in a parallel configuration so to facilitate environmental uniformity of the inter-connected growth surface tiers. The unique design also facilitates a fine-tuning of optimized gas and media flow to each cell growth level within the vessel stack. The unique architecture facilitates media refreshment at conveniently scheduled intervals and/or allows constant perfusion from a media reservoir that can be replenished without interrupting the cell proliferation rate. The perfusion rate, nutrient medium condition, CO2 content and osmolality can be controlled to optimize the desired cell proliferation rate, thereby improving cell quantity, quality, and viability. There are case studies, since 2008, (Zhu, M. et al) that have been published citing specifically that if pCO2 and osmolality increase in cell culture that cell viability will correspondingly decrease. In the paper published by Zhu et al, cell viability dropped 20% at 140 mm pCO2 and 400 mOsm/kg. The parallel interconnected vessels may be environmentally and/or nutritionally optimized, in real time, for cell quantity, quality, viability, and protein/antibody production.

An objective of this application is to produce cell growth vessels that are architecturally identical and capable of being stacked in a parallel arrangement or facilitate the sharing of all the elements of a common residence environment as well as each vessel being connected by a compound manifold to facilitate delivery of identical volumes of a pH controlled nutrient medium from a common source. A second manifold is positioned to facilitate delivery of physiologically identical blends of oxygen and nitrogen, as required by the cells in residence, to each individual vessel of the stack. A third manifold is positioned to remove spent media and excess gas from each vessel in identical quantities under identical conditions at identical intervals. Furthermore, an objective of the stacked growth surfaces is to increase the surface area upon which the living cells can reside while maintaining the required footprint of the stack and, at the same time, keeping the cubical space requirement of the entire structure at a minimum.

Another aspect of the application is to provide a cell growth vessel stack system for unattended addition of fresh cell growth nutrient medium as required for the metabolic and physiological needs of living, proliferating, and secreting cells. The fresh nutrient medium would be held in common reservoirs with a capacity of 500 cc, more or less if required, and would provide the necessary devices for pre-gassing the fresh media, within each media reservoir, for the purpose of maintaining a normal pH as required for each different cell type or cell line. Presently, the pH target for the nutrient medium is most usually achieved with the addition of 5% CO2 to the cell culture incubator, as a residence gas, which is then equilibrated into each vessel, within the incubator, having a slightly loosened closure. The residence gas of the incubator equilibrates into the head-space of each cell culture vessel but then must be transferred into the nutrient culture medium that is bathing and feeding the cells in each cell culture vessel. There is the possibility that each individual culture vessel may not achieve exact and precise transfer of head-space gas into the nutrient medium because of slight variability of conditions between the individual culture vessels. Differences possibly include vessel location within the incubator, slight differences of media volume within each vessel, vessel closure differences, etc. In addition to the possible variables of the gas equilibration from the incubator into the head-space of each individual culture vessel, the transfer rate within each individual vessel may vary. Gas transfer from the head-space into the liquid nutrient medium is not the most efficient way to achieve the precise oxygen and carbon dioxide concentration within each cell culture vessel. By pre-adjusting the pH and oxygen concentration within each media reservoir, with a required gas mixture, it will ensure that each vessel in the stack will receive nutrient media with precisely the same gas and pH conditions.

The stack system herein described, with two fresh media reservoirs, one pre-gassed with 5% CO2, and the second reservoir either not pre-gassed or gassed with the oxygen content desired but with zero CO2, will enable the media for infusion to be blended to achieve any CO2 content between zero and 5% and thus obtain exactly the media pH required, achieved, and verified by using feedback information provided by the integral optical sensors for pH and dissolved oxygen. Such a system will facilitate the exact titration of the combined reservoir contents to achieve an exact and precise condition of the nutrient media delivered to each individual culture vessel in the stack in real time. Such a system will eliminate estimating the nutrient condition as a requirement of gas transfer from the head-space and also eliminate potential differences of oxygen uptake rate between the individual vessels.

Another important aspect of the application is to provide a simple, fast, exact, and precise method of media replenishment for management of culture lifetime and cycle times.

One aspect of the application relates to the use of an oxygen-permeable and liquid-impervious membrane to control the amount of oxygen from the vessel environment that penetrates into the cell attachment matrix. By using this method of providing oxygen at the cell attachment matrix, the barrier of the liquid medium between the oxygen of the head space and the cell attachment sites on the growth matrix is eliminated. The exact concentration of oxygen, selected to satisfy the optimum conversion of a carbon source, e.g. glucose, to energy during the metabolic process, is easily satisfied. Such an arrangement for providing oxygen at the adherent cell attachment site, and residence site of the cells upon the growth matrix, would eliminate the concern of obtaining sufficient oxygen transfer from the head space through the media barrier to the cell attachment site.

Another aspect of the application is to provide a cell growth vessel stack system that possesses the flexibility to include any of several different growth and cell attachment matrices. For example FEP Teflon™ is biocompatible and may be either plasma etched or Corona Treated, on one or both sides, to produce a hydrophilic surface that is cell friendly. The etching provides a cell growth surface for attachment of adherent cells. A growing variety of cell growth matrix membranes are also available for cell attachment. There are membranes that are oxygen permeable but liquid impervious. There are borosilicate fiber membranes, quartz fiber membranes, and high purity glass membranes each having specific features that may provide certain advantages when used as a cell attachment matrix in cell culture vessels. A very thin silicone elastic membrane is highly permeable to oxygen, very durable, cell friendly, has stable physical properties and therefore is the preferred matrix.

Another objective of the application is to provide a method for the easy removal of cells from a cell growth vessel stack system for washing and further use. The accepted practice for removing adherent cells from the growth surface of cell culture vessels is to use an adjusted concentration of trypsin added after desired culture yield has been achieved and then somewhat vigorously shake the culture vessel to release the cells. That release technique may not be gentle enough to recover cells with the confidence that there will be no cell damage. In embodiments of this application, the cell matrix may be lifted away from the cell growth vessel stack system with the cells intact, wherein the cells can then be released from the cell growth matrix with little or no physical stresses applied.

Another objective of the application is to provide a method for monitoring the conditions of the cell culture media with the use of optical sensors. The optical sensors are attached in direct contact with the cell nutrient media, near the cell attachment site, for continuous monitoring of the dissolved oxygen (DO), pH, glucose etc. Such optical sensors may then be excited using specific wavelengths of excitation light through the transparent vessel wall. The emission frequency that evolves from the excitation phase will also pass through the vessel wall for external reception and interpretation. These sensors may contain ratio-metric pH sensitive dyes, such as 1-hydroxypyrene-3,5,7-sulfonic acid (HPTS), or may contain a fluorescent oxygen-quenching construct to measure DO. This invention also allows for a luminescence detector to be inserted or removed from the stack of vessels without disturbing the ongoing culture process. One sentinel detector may be located in position on one vessel for constant sentinel monitoring or the detector may be moved from vessel to vessel for sequential monitoring or a plurality of detectors may be inserted to detect signals from more than one or all vessels simultaneously.

A further objective of the present application is to provide a method for controlling the pH and oxygen concentration of a cell growth culture by monitoring the data, provided by the resident sensors, and then either increasing or decreasing the mixture and/or flow rate of the gas. Media exchange intervals and/or perfusion rates can be monitored and controlled to provide the optimum energy conversion for robust cell proliferation and/or for optimum protein and/or antibody cell secretions.

An important objective of the invention is to provide a design and process that, upon delivery of nutrient media and gas into a common pre-distribution chamber, will separate the two phases (gas and liquid) so the liquid portion will increase somewhat in depth until it spills uniformly over a very low moat that, by design, will deliver the liquid evenly onto the cell growth matrix and have it flow uniformly and progressively over the growth matrix and adhered cells. The gas phase will fill all additional space within the vessel. The gas thereby reaches the matrix-adhered cells through the gas permeable membrane at the site of the attachment of the matrix-adhered cells. This process will obviate the need for the gas to transfer from head space through the resistant liquid medium before reaching the cell attachment site. It will also ensure that the desired oxygen concentration required for optimum cell growth will not be reduced by partial absorption of oxygen into the liquid medium or if CO2 becomes elevated enough to change the saturation of oxygen in the nutrient media.

It is also an objective that each stack of four identical vessels receive identical quantities of seed cells at the same time and under the same conditions.

As aerobic cells grow and proliferate, using nutrients absorbed from the nutrient cell culture medium, a series of chemical actions occur as a result of the metabolic process. Such reactions include the citric acid cycle (CAC) which is also known as the tri-carbolic acid cycle (TCA) or the Krebs cycle. The Krebs cycle consumes acetate and water and reduces NAD to NADH and thus releases carbon dioxide. If the nutrient medium becomes saturated with carbon dioxide, it is probable that some CO2 will escape from the nutrient medium into the resident gas space within the stacked culture vessels. To ensure against change in the resident gas from the addition of carbon dioxide gas, this invention includes the provision for adding a small cassette containing a carbon dioxide absorbent such as AMSORB® which is a CO2 scavenger.

In an exemplary embodiment, a method of culturing involves: attaching cell cultures onto a plurality of cell growth matrixes, placing the cell growth matrixes into a plurality of cell growth modules, arranging the cell growth modules to be parallel with one another in a cell growth vessel stack system, adding cell culture media to cell growth modules, attaching a residence gas delivery manifold onto the cell growth vessel stack system, monitoring the cell culture environments within each cell growth module, and recovering the cell cultures from the cell growth vessel stack system after the cell cultures have reached a specified cell density.

In some embodiments, the method further comprises placing the plurality of cell growth modules into an incubation device to regulate the temperature of the cell growth vessel stack system.

In some embodiments, the cell culture environments are monitored through the use of optical fiber sensors, light pipes possessing a sensor, a sensor reader, a gas sensor or any combination thereof.

In some embodiments, the cell culture environments are monitored through the use of a pH patch sensor that generates a ratio-metric response.

In some embodiments, the cell culture environments are monitored through the use of a fluorescent oxygen-quenching patch sensor.

In some embodiments, the cell cultures are recovered from the cell growth vessel stack system after the cell cultures have reached a cell density that covers between 60% to 100% of the cell growth matrix.

In some embodiments, the method further comprises prepping the cell culture media before adding such media to the cell growth modules.

In some embodiments, during the prepping step the pH and DO levels of the cell culture media are adjusted, wherein the adjusted pH value is between 6.5 and 7.5, and wherein the adjusted DO level is between 0.1% and 20%.

In some embodiments, the media preparation consists of using two media reservoirs, wherein a first reservoir is prepped with the addition of 5% carbon dioxide to activate a bicarbonate/carbon dioxide buffering system to stabilize the media pH to between 6.5 and 7.5; and wherein a second reservoir is not prepped with carbon dioxide so that media from each reservoir may be blended prior to introduction into the cell culture vessels to compensate for excess carbon dioxide produced within the culture vessels due to glycosylation.

In some embodiments, method further comprises recovering metabolites, proteins, antibodies, exosomes and any combinations thereof from the cell cultures.

In an exemplary embodiment, a cell growth vessel stack system includes: a plurality of cell growth modules arranged in parallel with each other, wherein the cell growth modules further comprise a cell growth matrix, for impregnation with cell cultures, gas inlets, media inlets, and reception area for seed cell inoculums, at least one fresh media reservoir connected to the media inlets of the cell growth modules, and a spent media reservoir connected to the media outlets of the cell growth modules, wherein the cell growth modules of the cell growth vessel stack system share a common cell culture environment.

In some embodiments, the cell growth modules further comprise a removable tray.

In some embodiments, the removable tray further comprises a cell growth matrix and a liquid-impermeable, gas-permeable membrane.

In some embodiments, the system further comprises a rotator or rocker that holds the cell growth vessel stack system and provides lateral or oscillating movement to the cell cultures.

In some embodiments, the system further comprises an incubation device that houses the plurality of cell growth modules.

In some embodiments, the system further comprises a media/gas distributor module at the top of each cell growth vessel of the stack.

In some embodiments, the media/gas distributor module possess a moat, a liquid media inlet and a residence gas inlet.

In some embodiments, the removable tray further comprises a drop bridge.

In some embodiments, the cell growth modules further comprise residence for a CO₂ scavenger.

In some embodiments, the system further comprises a closed media pumping system comprising syringe and peristaltic pumps.

In some embodiments, at least one fresh media reservoir further comprises a sparge gas inlet and a sparge gas outlet.

In some embodiments, the system further comprises a cell residence gas inlet.

### BRIEF DESCRIPTION OF THE DRAWINGS

FIG. 1 depicts a lateral schematic interpretation of a stack of four identical cell culture vessels that are commonly interconnected in parallel configuration to a gas manifold that delivers premixed residence gas to the cell culture matrix head-space via a media/gas separation and delivery chamber that is a component of each vessel. A second manifold, compound in design, is for delivery of fresh nutrient media to the media/gas separation chamber for blending and/or delivery to the cell growth matrix. A third manifold is a conduit for the removal of spent nutrient media and excess head-space residence gas. Two syringe pumps and one peristaltic pump cooperate to deliver pH adjusted nutrient media to the identical culture vessels and to remove spent media and excess residence gas simultaneously from each vessel. Two fresh media reservoirs are for holding sterile nutrient media prior to delivery to the parallel and interconnected cell culture reaction vessels. A sealed spent media vessel receives both spent media and excess residence gas and provides a filtered vent to release sanitized gas to the environment.

FIG. 2 illustrates the Central Housing of an individual cell culture vessel from the stack. It also shows the clear plastic sensor holder plate in a flipped-up orientation to better visualize the sensor light pipes and the means and orientation required for insertion of the sensor holders through the two openings on the top surface of the Central Housing. The compartment shown at the uppermost right of the Central Housing is the Media/Gas Distribution Compartment and the media and gas entry ports. The transparent top enables visualization of the internal components of the compartment.

FIG. 3 is a representation of the components of the Media/Gas Compartment in greater detail for visualization.

FIG. 4 illustrates the positional relationship between the Central Housing and the optical sensor reader and the plastic alignment holder for the light emitting/luminescence detector. It demonstrates the multifunctional purpose of the alignment holder as a component that will function not only to precisely align, allow removal of the emitter/detector if desired, and precisely re-align the emitter/detector device over the optical sensors and also functions as an architectural foundation for the central housing of the vessel module to be stacked and aligned immediately above the module illustrated. The emitter/detector holder functions to allow removal and re-installation of the emitter/detector device without moving or disturbing any other vessel of the stack. The primary function of the emitter/detector is to excite the optical sensors, receive the luminescence response to that excitation, condition the luminescence signal in the firmware housed therein and transmit the conditioned signal to a laptop computer for interpretation of the conditioned signal. The emitter/detector is controlled by laptop computer software that is designed to cause the emitter/detector to activate upon command or at specifically programmed recurring intervals. The signal received from the emitter/detector by the laptop computer is the basis for software response such that the cell culture environment may be adjusted for optimum condition by starting or stopping liquid media pumps or turning-on or turning-off gas flow or adjusting the mixture of the residence gas from time-to-time to automatically maintain the optimum inner environment of the culture vessel module as programmed into the software.

FIG. 5 shows a Front View, Lateral View, and Top View of the Internal component I that provides support for the cell culture matrix tray and also holds the matrix tray in position under the optical sensors to allow the collection of sensor derived information from as close to the cell attachment layer as possible thereby enabling real-time monitoring of the metabolic process and immediate response to any deficiencies of the nutrient medium condition. The matrix tray holder and positioning device also provides a small rectangular receptacle for a small cassette for holding AMSORB® which is a non-CO releasing CO2 scavenger used primarily in anesthesiology. The AMSORB is to ensure that any CO2 produced during the citric acid cycle of cell biology and out-gassed from the liquid medium as a result of hyper-saturation does not impair cellular respiration.

FIG. 6 is a Top View, Side View, and Front View of the Inner component II which is also the Matrix Tray for holding the silicone cell growth matrix which overlays a gas diffusion medium. The silicone growth matrix will provide for attachment of adherent cells which will then be bathed in fresh nutrient media and held therein during the growth and metabolic process time until the time comes for harvesting the cells or collecting the secretions of the cells. The row of identical geometric openings in the bottom surface of the tray provides passage for residence gas to fully saturate the gas diffusion medium and permeate the silicone growth matrix to reach the cell attachment site.

FIG. 7 Illustrates a scaled-down drawing of the matrix tray (Inner component II) with a fabric mesh material (Inner component III) overlaying the gas slots as shown in FIG. 6.

FIG. 8 Illustrates, in another scaled down drawing, how the gas permeable silicone membrane, upon which the cell growth media will rest, overlays the thin fabric mesh and adheres tightly to the periphery of the inner tray surface.

FIG. 9 depicts a holder and positioning module for light emitter and detector device having the purpose of emitting an excitation light and collecting the luminescence response.

FIG. 10 is an end cap that snaps into place over the open end of each central housing thereby providing for complete isolation of each vessel of the stack from the outside environment

FIG. 11 is an external view of a completely assembled cell culture vessel of the stack.

FIG. 12 is a schematic representation of a stack of four cell culture vessels that will become the integrated group of parallel and interconnected cell culture vessels.

FIG. 13 depicts an embodiment of the drop bridge.

### DETAILED DESCRIPTION OF INVENTION

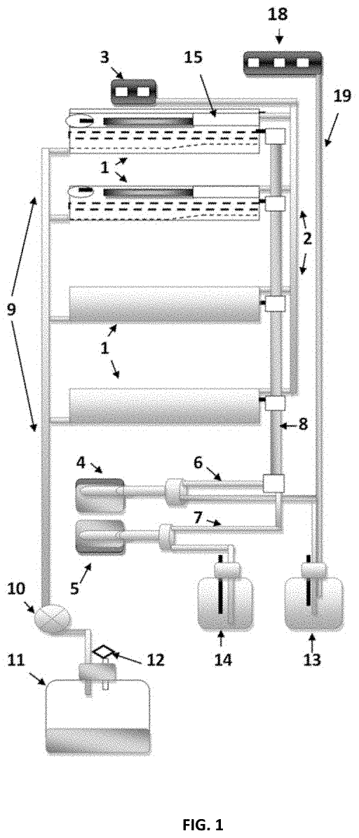

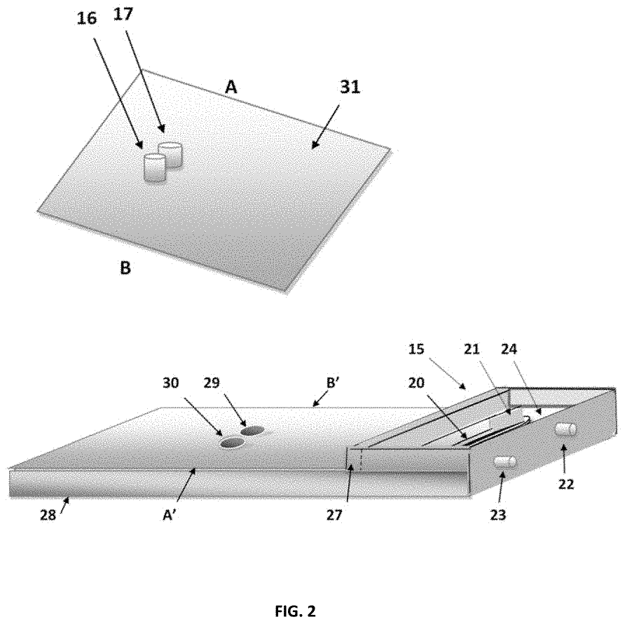

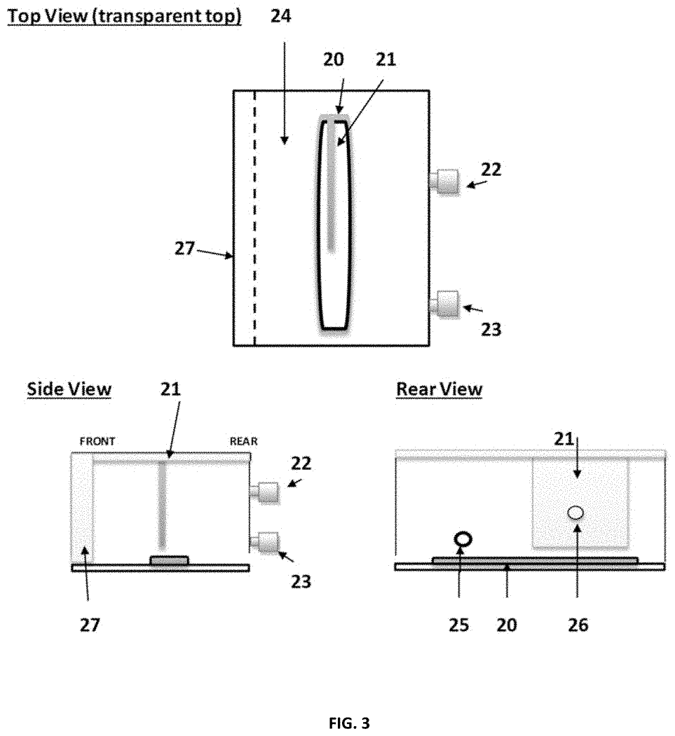

FIG. 1 depicts a lateral interpretation of a stack of four identical cell culture vessels 1 stacked and connected to a residence gas delivery manifold 2 and a gas control device 3. Two syringe pumps 4,5 deliver fresh nutrient media to the stacked culture vessels via media delivery lines 6,7 through compound liquid delivery manifold 8. Spent media and excess residence gas are removed from the stacked vessels through a spent media waste manifold 9 as a result of the action of a peristaltic pump 10 which deposits both spent media and excess residence gas into a sealed waste receptacle 11 that allows the excess resident gas to exit to the atmosphere only after being sanitized through a filtered vent 12. One syringe pump 4 pulls nutrient media that has been pH adjusted with the addition of 5% CO2 from fresh media reservoir 13 and delivers that media through a delivery line 6 to each vessel of the stack via a compound manifold 8. The second syringe pump 5 pulls fresh media having no CO2 addition from fresh media reservoir 14 and delivers that media through a delivery line 7 to the stacked vessels via compound manifold 8. Fresh media being delivered via compound manifold 8 is diverted to each one of the vessels in the stack through the single media entry port of each vessel and thereby enters a common media/gas compartment 15 of each individual vessel. The media delivery may be 100% from the reservoir 13 or 100% from the reservoir 14 or a custom blend of the media from each reservoir as called for by control software that is responding to pH and DO sensor information from optical sensors 16,17 (FIG. 2) that are positioned inside the culture vessel at the peri-cellular level of adherent cells residing on the cell growth matrix. One gas control device 3 is a mass flow controller that directs custom blended air and nitrogen through the residence gas manifold 2 for delivery to each vessel via a gas entry port into each media/gas compartment 15. The second mass flow controller 18 directs custom blended nitrogen, oxygen, and carbon dioxide, via a gas sparger line 19 to the fresh media reservoir 13. The fresh media in the gas/media compartment 15 is delivered into the cell growth part of each vessel with the media flowing uniformly over a very low moat 20 (FIG. 3) causing the media to flow evenly onto the cell growth matrix. The gas in the gas/media chamber 15 is distributed uniformly throughout each cell culture vessel 1 and fills all unoccupied space.

The unique architecture allows one to facilitate media refreshment within the vessel when indicated by the optical sensors or at conveniently scheduled intervals or for constant perfusion from a media reservoir that can be replenished without interrupting cell proliferation rate. The perfusion rate may be controlled to optimize the desired cell proliferation rate. For example, as the perfusion rate is increased, cell proliferation increases and the quantity of viable cells will increase. As the perfusion rate is slowed, the proliferation rate of cells also slows and the protein/antibody secretion output of the cells will increase. Therefore the parallel interconnected vessels may be environmentally and/or nutritionally optimized for either cell quantity and viability or protein/antibody production. The cell growth vessel stack system has been designed especially for adherent cells and/or for cell differentiation but may be used to grow cultures of cells commonly used by those in the art. Stem cell differentiation would be one use of such a system.

FIG. 2 represents an external view of the Central Housing 28 of one of the vessels from the stack. The Media/Gas Distribution Compartment 15 is located as an integral part of the Central Housing. Two plastic light pipes with sensors 16,17 are molded into a clear plastic plate 31 that is flipped over and oriented atop the Central Housing so that sides A and B correspond with A′ and B′ of the Central Housing and light pipes with sensors 16,17 are inserted into respective holes 29,30. One inlet 22 is for gas introduction and an inlet 23 is for media introduction. There is a three-sided canopy-like projection 27 that is visualized better on FIG. 3. Components 20,21,24 are also better detailed in FIG. 3.

FIG. 3 shows a Top View, a Side View, and a Rear View of the Media/Gas Distribution Module. Residence gas enters the media/gas distribution module through an entry port 22 and distributes to all unoccupied vessel space through the opening surrounded by the low moat 20. Fresh media enters the media/gas distribution module through a second entry port 23 at a slow flow rate that enables the liquid to cover the entire area of the floor 24 and rise slowly and uniformly until it spills over the walls of the low moat 20 and is distributed uniformly across the width of the cell growth matrix which resides on a matrix tray just inferior to the moat 20 opening. There is a baffle 21 that extends downward from the top of the media/gas enclosure. The residence gas port 22 also functions as a seed cell inoculation port. After liquid media and residence gas have been introduced and the media, gas, and temperature of the vessel have stabilized, seed cells are inoculated through the residence gas port 22 using a syringe and long blunt-end needle or thin cannula that is inserted until the long needle comes to rest against the baffle 21. As seed cells are gently inoculated and cling against the baffle they flow downward through the opening of the moat 20 and distribute into the fresh nutrient medium in the matrix tray. Uniform distribution of the cells is aided by a very slow rotary motion of the culture vessel that is either provided by the incubator module itself or an accessory device that will maintain very slow rotary motion. There is a three-sided (top, right, and left) canopy-like projection 27 at the front of the media/gas distribution module that provides a receptacle for one end of a sensor detector holder and guide. The holes 25,26 in the rear wall are for Luer fittings 23,22 that will enable media and gas entry and distribution.

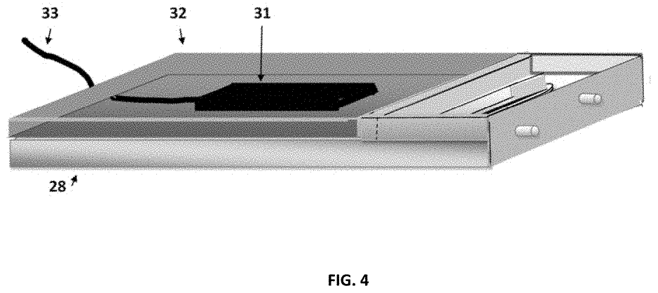

FIG. 4 is a schematic view of the Central Housing 28 of FIG. 2 depicting an Optical Sensor Detector 31 residing in a Sensor Detector Holder 32 that fits atop the central housing 28 and provides an architectural foundation with profile and dimensions required for stability when stacking vessel upon vessel. A USB connecting wire 33 is shown exiting the vessel for connection of the sensor emitter/detector to a power source i.e. a laptop computer. Once installed, the emitter detector 31 can be easily removed and precisely repositioned, if desired, without disturbing any of the vessels in the stack.

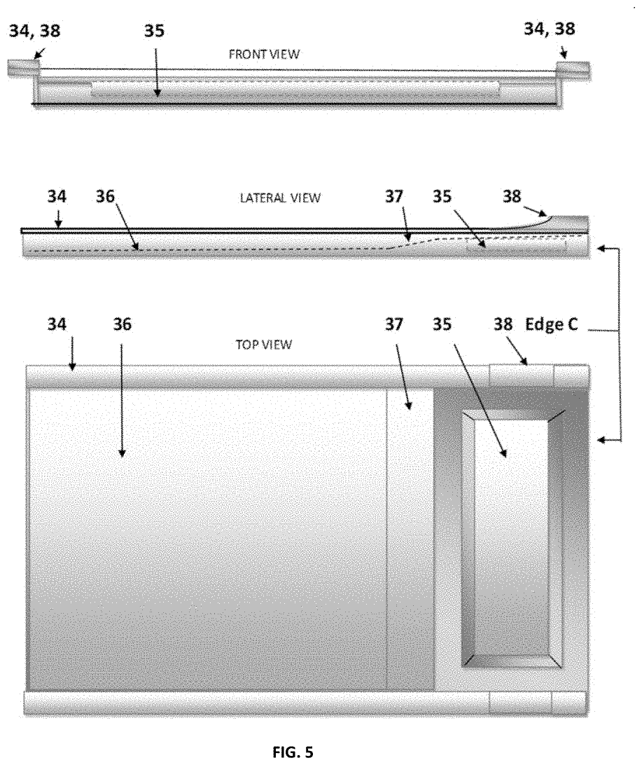

The internal structural components of the central housing of each cell culture vessel module, when assembled together within each of the stacking modules, provide the framework basis that enables the establishment of any chosen cell line and its given process requirements. The assemblage of internal components co-operating together enables the physiology of ex-vivo cell cultures to mimic in-vivo cell physiology. The structural components are designed to support the dynamic physiological process and enable the metabolic waste products of cell metabolism to clear from the cell culture vessels without creating a situation wherein viable cells are left to bathe in their own waste metabolic products. FIGS. 5, 6, and 7 will provide visualization of the internal components and the interconnectivity of each with the others.

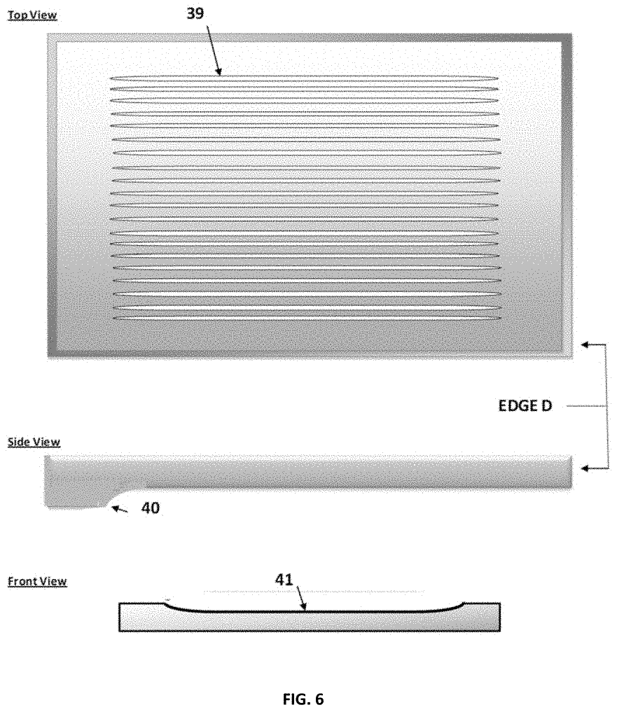

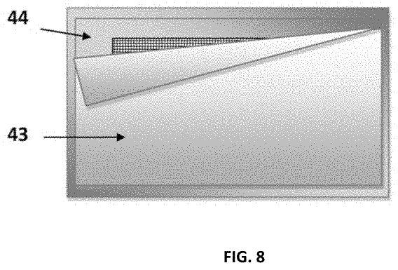

FIG. 5 shows a front view, a lateral view, and a top view of an Inner component I of four inner components of the central housing 28 (FIG. 2). Inner component I, with Edge C leading, slides into the central housing until it comes to rest at the rear inner wall of the central housing 28 and resting therein will have three functions. Inner component I (FIG. 5), serves as a foundation and position guide for Inner component II (FIG. 6) and will guide the inner component II inward on the lateral runners 34 and upward until Inner component II comes to rest atop the guide 38 which will be one of two positioning guides that will position the matrix tray so that the optical sensors 16,17 projecting through the top of the central housing 28 will rest immediately above the silicone cell growth matrix 43 of FIG. 8 and the attached layer of cells growing thereon. Another positioning guide 40 will be an integral part of the matrix tray (FIG. 6). A second function of Inner component I (FIG. 5) will be to accept the spent media overflow from the cell growth matrix and to collect such spent media in the reservoir portion 36 of Inner component I such that the media may be removed completely from the holding reservoir 36 of the central housing. The third function of Inner component I will be to hold a cassette of AMSORB granules in a compartment 35 that will facilitate the scavenging of unwanted CO₂ from the residence gas. A ramp 37 connecting the cassette compartment 35 with the reservoir 36 is to prevent spent media from collecting and backing up into the compartment 35.

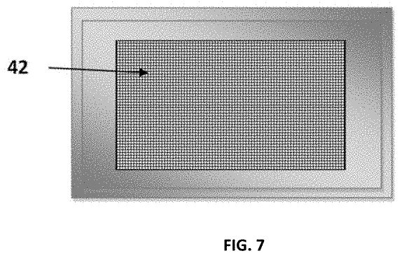

FIG. 6 represents an Inner component II of the four inner components. It is also the Matrix Tray that holds the silicone cell growth matrix 43 (FIG. 7) on which the process of cell culturing advances. The geometric slots 39 aligned on the bottom of the tray provide access for the residence gas containing the desired oxygen content to reach the growing cells at the cell attachment site by first fully saturating and then diffusing through the gas diffuser layer 42 and then permeating the silicone cell growth matrix 43 (FIG. 8) to reach the cell attachment site. The matrix tray (FIG. 6) is inserted, with Edge “D” leading, into the central housing 28 of each cell culture system module until it seats itself atop the matrix tray positioning guide 38 of Inner component I and at the same time coming to rest upon positioning guide 40 of the matrix tray of FIG. 6. Positioning guides 38,40 affix the matrix tray in its uppermost position against the inside of the upper surface of the central housing 28 thereby enabling the optical sensors 16,17 to collect sensory response from the cellular attachment sites. The front profile of the matrix tray (FIG. 6) illustrates a sculptured spillway 41 of the front surface of the tray which is a cut-out for the purpose of allowing spent media to spill over to the spent media reservoir portion 36 of inner component I and subsequently evacuated completely from the culture vessel.

FIG. 7 illustrates how the thin fabric mesh inner component III gas diffuser 42 is placed upon the matrix tray of FIG. 6 and covers the geometric slots 39. The geometric slots allow the free passage of the residence gas and as the mesh becomes saturated with oxygen-containing gas that diffuses onto the gas permeable silicone membrane 43, shown in FIG. 8, where the oxygen will penetrate the silicone membrane 43 at the cell attachment site. The oxygen of the residence gas, at the cell attachment site of the silicone membrane 43 is for cellular use for energy conversion of the nutrient glucose of the nutrient medium.

FIG. 8 illustrates how the oxygen permeable silicone membrane 43 overlays the fabric mesh gas diffuser medium. The very thin silicone membrane 43 by virtue of its properties to cling to smooth surfaces will hold in-place by attraction to the smooth perimeter 44 of the matrix tray. The matrix tray (FIG. 6), with a depth of 4 mm, more or less, enables the silicone membrane 43 to provide adherent cell attachment while being bathed in liquid nutrient medium that is present to fulfill the physiological requirements of cell metabolism. As cell metabolites are released into the nutrient medium, the optical sensors 16,17 will track the metabolic process and signal the control software when it is time to allow the spent medium to spill over the spillway 41 of the matrix tray to the media collection reservoir 36 of Inner component I (FIG. 5) from where it will be subsequently removed completely from each vessel of the stack. The removal of spent medium over the spillway 41 of each matrix tray may be assisted by the temporary, less than one minute duration, tilting of the stack of vessels about two degrees upward at the fresh medium entry 23 end of the stacked vessels just prior to when the syringe pumps 4,5 initiate the process of re-filling the cell growth chamber of each matrix tray with fresh nutrient medium from either or both fresh media reservoirs 13,14. The matrix tray of each cell culture vessel has a total capacity of 60 cc before the media spills over the spillway 41 of the matrix tray. Generally, the syringe pumps 3,4 are programmed to deliver a total of 40 cc, two-thirds of total capacity, for the residence of the growing cells. Then, based on sensor 16,17 response, the software program can call for an additional 20 cc of fresh media if/as required to maintain optimum physiological condition of the nutrient medium. Anything over 60 cc in the matrix tray will spill over the spillway 41 as spent media. Four vessels of the stack will require a recommended total of 160 cc of working volume of nutrient medium for a combined 115,200 square millimeters of cell attachment area.

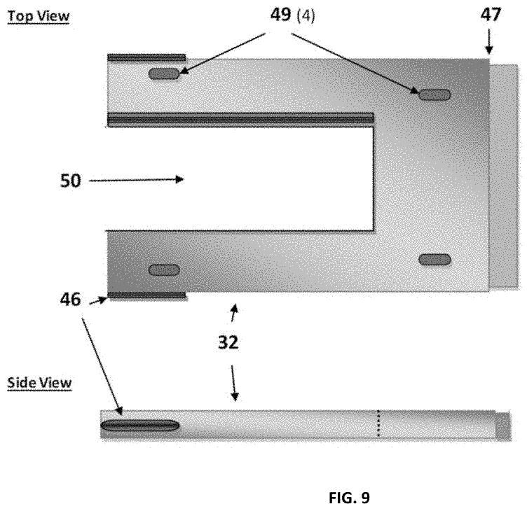

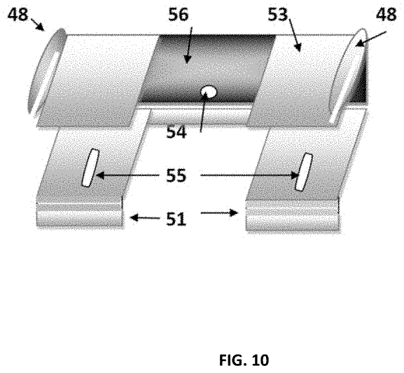

FIG. 9 depicts the Sensor Holder/Positioning Component 32 which, when in place atop the central housing 28 of each vessel of the stack enables the sensor detector/reader 31 to slide precisely, via the detector/reader guide 50, into position over the optical sensors 16,17 that indwell the parallel and interconnected vessels of the stack. In use, the stack may operate with one sentinel sensor detector/reader 31 which may remain in one position or be moved from vessel to vessel if/as desired or, alternatively, one sensor detector/reader 31 may be placed in position on the central housing 28 of each vessel of the stack and monitor all vessel sensors simultaneously. The architectural features of the component 32 are such that one end is rabbet cut on three sides and makes a rabbet joint 47 with the receptor canopy portion 27 of the media/gas distribution component of central housing 28. The rabbet joint 47 along with the outer front wall of the media/gas distribution compartment 15, the canopy 27, and the top surface of the central housing 28, and the components of the end cap 53 (FIG. 10) cooperate to completely immobilize component 32 and thereby maintain strict alignment of the emitter/detector 31 as it slides into channel 50 and comes to rest at its innermost position.

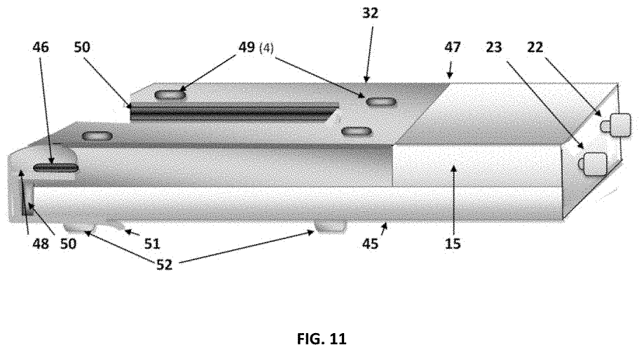

FIG. 10 shows the components of an anodized aluminum end cap 53. As the end cap snaps into place over the single end opening of the central housing 28, the adhesive-backed gasket 56 completely isolates the fully assembled vessel from the outside environment. The end cap features two side-guides with slots 48 that fit over side-ears 46 of component 32 (FIGS. 9 and 11). Two open slots 55 snap snugly over projections 52 (FIG. 11). Finger release tabs 51 are used to release the end cap 53 for removal. The single hole 54 in the back wall is for ⅛-27 NPT luer fitting for connection to spent media removal manifold 9 (FIG. 1).

FIG. 11 depicts an external view of an assembled vessel 45, showing the assembly relationship of following components: Component 32 (emitter/detector 31 holder), central housing 28 (FIGS. 2 and 4), and the end cap 53. Also detailed are the four stacking receptors 49 for insertion of stacking projection tabs 52 and guide.

When the sensor detector/reader holder 32 is in place atop the central housing 28, an end cap 48, most conveniently fabricated of anodized aluminum and having two convenient tabs 51 (FIG. 10) with slots 55 that receive tabs 52. The end cap 53 is designed to snap into place over the single open end of each central housing 28 and hold, in a locked together assemblage, of all components of each cell culture vessel of the stack. One adhesive backed, polyurethane gasket 56 is adhesively affixed to the inside surface of the end cap 48 such that when the end cap is snapped into place, against the open end of each central housing 28 of the stack, the entire system of stacked culture vessels 1, plumbing 6,7,8,9, manifolds 2,8,9, media reservoirs 13,14, syringe components of syringe pumps 4,5, and the spent media reservoir 11, all depicted as the parallel and interconnected stack of vessels of FIG. 1 are completely sealed and isolated from the external environment. The entire sterile, closed system of components assembled together may be placed in any constant temperature incubator designed for cell culture purposes but for the convenience of a complete “turn-key” system, a rotating plate incubator has been modified to conveniently operate with two stacks of the herein invented stacked vessels thereby extending the closed system to include all components necessary to successfully complete virtually any and all upstream experimental and production processes and some very specific downstream processes

FIG. 11 is also an illustration of the external appearance of a completely assembled cell culture vessel module that depicts all of the interlocking assemblage and alignment devices that facilitate the creation of a parallel and interconnected cell culture vessel system. Integral to the central housing are four alignment tabs 52 that fit into the four alignment receptacles 49 of an adjacent and inferiorly positioned cell culture vessel module. The detector holder component 31 is held firmly in place within a junction receptacle 47 and abuts the media gas distribution compartment 15 of the central housing 28, facilitated by slots integral to the end cap 48 that mate with groove counters 46 that are sculpted into the detector holder 32. The emitter/detector 31 is precisely positioned over sensors 16,17 by sliding into a channel 50 of the holder/positioning module 32. The residence gas entry port 22 and the fresh medium port 23 are the entry portals for the introduction of nutrient liquid media, residence gas, and inoculated seed culture cells into each vessel of the stack. All liquid, gas, and cells are introduced to the system through the media/gas distribution module 15. All components are separated before entry into the central housing 28, and then uniformly and precisely distributed to the proper residence location for each phase of the system where they reside in full cooperation with each other for the achievement of successful cell culturing of the variety of mammalian cells of interest in an automatically optimizing culture system controlled and facilitated by firmware and operator programmed software that relies on data generated by indwelling sensors 16,17, for collection by the sensor detector/reader 31 and then interpretation and real-time response to cell requirements for production of viable cells and quality metabolic conversions and secretions and/or shedding of proteins, antibodies, exosomes and excipients.

The equilibrium of the inner environment of each cell growth vessel of the stack is maintained between the incoming pressure of the residence gas combined with the gentle pushing of the fresh media and the pressure relief that occurs with the action of the peristaltic pump 10 as called for by the software program. The system of the invention herein described accomplishes precisely prescribed inner equilibrium without a single shut-off valve, check valve, or pressure relief valve. The process is controlled by a software program with feedback information from indwelling sensors that permits the operator to intervene if desired.

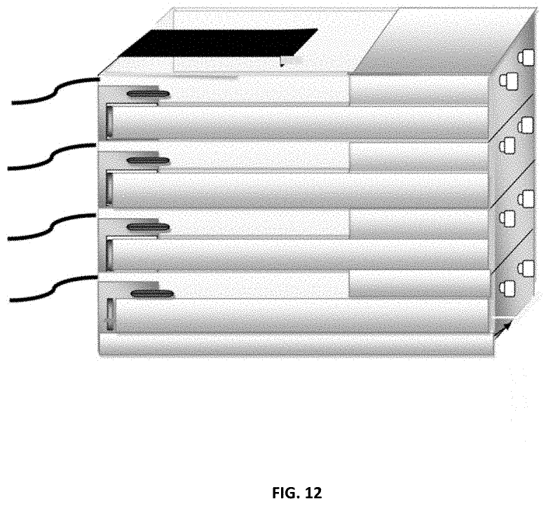

FIG. 12 depicts a plurality of growth vessels of the stack system as they will be stacked and placed into an incubator that will provide a desired incubation temperature and, if desired, gentle low speed rotation or rocking motion. A slow rotary motion of 20 RPM, more or less, is used when inoculating seed cells into the system. The slow rotary motion enhances the uniform distribution of seed cells in the liquid medium. The stacking of the plurality of cell growth modules allows an increase of the surface area upon which the living cells can reside and grow while maintaining the footprint of a single vessel and minimizing the cubical space requirement of the entire stack.

The mass flow controller 3 that is depicted in FIG. 1 is for blending pure nitrogen with atmospheric air to achieve a desired oxygen content of the resident gas between 0.1% and 20%. The Mass flow controller 14 depicted in FIG. 1 is for blending nitrogen, oxygen, and carbon dioxide that is used for adjusting the pH content and, if required, the oxygen content of the fresh nutrient media of reservoir 13 (FIG. 1). When the nutrient medium within the reservoir 13 has achieved the desired pH, oxygen content, and temperature, as verified by indwelling sensors, the media is delivered by action of the syringe pump 4 to each of the stacked vessels via the compound manifold 8 at a rate that will allow the media to flow gently through the media/gas distribution compartment and uniformly across the width of the surface of the cell growth matrix. The flow rate and pressure of the fresh media is controlled by the two syringe pumps that respond to transmission of data indicating the condition of the environment of the cell growth matrix which includes pH and oxygen content information transmitted by the indwelling optical sensors as well as the culture vessel temperature information. The volume and depth of nutrient medium in each culture tray is calculated from the data output of each syringe pump. If the pH of the liquid medium within each vessel should drop below what is desired based on the type and nature of the cell culture, the second syringe pump 5 will titrate liquid media from fresh media reservoir 14 so as to adjust the pH back to normal as indicated by the optical pH sensor or sensors residing within the liquid media at the cell growth level.

Each matrix tray within each vessel of the stack will accept a depth of 3 mm of liquid. Anything more than 3 mm depth will spill from the matrix tray and will be collected by the spent media reservoir for immediate evacuation through the end cap to a sealed waste collection reservoir. As gas pressure within the sealed waste receptacle increases, it is released from the sealed waste receptacle through a sanitizing filter with 0.2 micron pore size. A 20 square centimeter tray area will hold 20 cc of liquid for each millimeter of tray depth.

The spent media and fresh media reservoirs may be molded containers, or any other glass or plastic reservoir or storage device commonly used within the art.

FIG. 4 shows an external embodiment of a cell growth module 32 which can be used as component of the cell growth vessel stack 1.

In some embodiments, the cell growth vessel stack 1 may comprise 1 to 10 cell growth modules 32. In other embodiments, the cell growth vessel stack 1 may comprise more than 10 cell growth modules. The cell growth modules 32 in the cell growth vessel stack 1 may be arranged in parallel but are inter-connected in such a way that each module 32 shares a common temperature and/or gas environment.

In some embodiments, the cell growth vessel stack system comprises cell growth modules that possess a drop bridge. A drop bridge can be used to hold a membrane in place within the cell growth modules. The membrane can be either a cell growth matrix, a liquid-impermeable gas-permeable membrane or any other membrane commonly used in the art. The drop bridge can be the same height as a removable tray within a cell growth module. The drop bridge may possess an opening in the center to allow easy examination of the cell culture environment in the removable tray. The drop bridge can be created from a polystyrene (PS) material, a polyethylene terephthalate (PET) material, a high- and low-density polyethylene (PE) material, a polyvinyl chloride (PVC) material, a polypropylene (PP) material or any combination thereof.

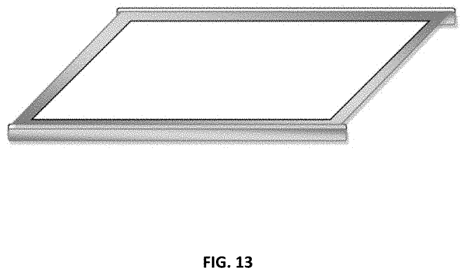

FIG. 13 depicts an embodiment of the drop bridge 47. The drop bridge 47 possesses an opening within the center to allow easy examination of the cell culture environment in the cell growth module.

It will be apparent to those skilled in the art that numerous modifications and variations of the described embodiments are possible in light of the above teachings of the disclosure. Other alternate embodiments may include some or all of the features disclosed herein. Therefore, it is the intent to cover all such modifications and alternate embodiments as may come within the true scope of this application, which is to be given the full breadth thereof. Additionally, the disclosure of a range of values is a disclosure of every numerical value within that range, including the end points.

We claim:

1. A method of culturing cells comprising:
attaching cell cultures onto a plurality of cell growth matrixes,
placing the cell growth matrixes into a plurality of cell growth modules,
arranging the cell growth modules to be parallel with one another in a cell growth vessel stack system,
adding cell culture media to cell growth modules,
attaching a residence gas delivery manifold onto the cell growth vessel stack system,
monitoring the cell culture environments within each cell growth module, and
recovering the cell cultures from the cell growth vessel stack system after the cell cultures have reached a specified cell density.

2. The method according to claim 1, wherein the method further comprises placing the plurality of cell growth modules into an incubation device to regulate the temperature of the cell growth vessel stack system.

3. The method according to claim 1, wherein the cell culture environments are monitored through the use of optical fiber sensors, light pipes possessing a sensor, a sensor reader, a gas sensor or any combination thereof.

4. The method according to claim 1, wherein the cell culture environments are monitored through the use of a pH patch sensor that generates a ratio-metric response.

5. The method according to claim 1, wherein the cell culture environments are monitored through the use of a fluorescent oxygen-quenching patch sensor.

6. The method according to claim 1, wherein the cell cultures are recovered from the cell growth vessel stack system after the cell cultures have reached a cell density that covers between 60% to 100% of the cell growth matrix.

7. The method of claim 1, wherein the method further comprises prepping the cell culture media before adding such media to the cell growth modules.

8. The method of claim 7, wherein during the prepping step the pH and DO levels of the cell culture media are adjusted, wherein the adjusted pH value is between 6.5 and 7.5, and wherein the adjusted DO level is between 0.1% and 20%.

9. The method of claim 7 wherein the media preparation consists of using two media reservoirs, wherein a first reservoir is prepped with the addition of 5% carbon dioxide to activate a bicarbonate/carbon dioxide buffering system to stabilize the media pH to between 6.5 and 7.5; and
wherein a second reservoir is not prepped with carbon dioxide so that media from each reservoir may be blended prior to introduction into the cell culture vessels to compensate for excess carbon dioxide produced within the culture vessels due to glycosylation.

10. The method of claim 1 wherein the method further comprises recovering metabolites, proteins, antibodies, exosomes and any combinations thereof from the cell cultures.

11. A cell growth vessel stack system comprising:
a plurality of cell growth modules arranged in parallel with each other, wherein the cell growth modules further comprise a cell growth matrix, for impregnation with cell cultures, gas inlets, media inlets, and reception area for seed cell inoculums,
at least one fresh media reservoir connected to the media inlets of the cell growth modules, and
a spent media reservoir connected to the media outlets of the cell growth modules,
wherein the cell growth modules of the cell growth vessel stack system share a common cell culture environment.

12. The cell growth vessel stack system of claim 11, wherein the cell growth modules further comprise a removable tray.

13. The cell growth vessel stack system of claim 12, wherein the removable tray further comprises a cell growth matrix and a liquid-impermeable, gas-permeable membrane.

14. The cell growth vessel stack system according to claim 11, wherein the system further comprises a rotator or rocker that holds the cell growth vessel stack system and provides lateral or oscillating movement to the cell cultures.

15. The cell growth vessel stack system according to claim 11, wherein the system further comprises an incubation device that houses the plurality of cell growth modules.

16. The cell growth vessel stack system according to claim 11, wherein the system further comprises a media/gas distributor module at the top of each cell growth vessel of the stack.

17. The cell growth vessel stack system according to claim 16, wherein the media/gas distributor module possess a moat, a liquid media inlet and a residence gas inlet.

18. The cell growth vessel stack system according to claim 12, wherein the removable tray further comprises a drop bridge.

19. The cell growth vessel stack system according to claim 11, wherein the cell growth modules further comprise residence for a CO₂ scavenger.

20. The cell growth vessel stack system according to claim 11, wherein the system further comprises a closed media pumping system comprising syringe and peristaltic pumps.

21. The cell growth vessel stack system according to claim 11, wherein at least one fresh media reservoir further comprises a sparge gas inlet and a sparge gas outlet.

22. The cell growth vessel stack system according to claim 11, wherein the system further comprises a cell residence gas inlet.
